(19) United States
(12) Patent Application Publication
(10) Pub. No.: US 2007/0129433 A1
(43) Pub. Date: Jun. 7, 2007

(54) Diphenylamine derivatives

(76) Inventors: Claude Lardy, Lyon (FR); Philippe Guedat, Montenois (FR); Lidia Caputo, Lyon (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/580,603

(22) PCT Filed: Nov. 17, 2004
(86) PCT No.: PCT/EP04/14916

§ 371 Date: May 25, 2006

(30) Foreign Application Priority Data
Nov. 27, 2003 (FR) 0313951

(51) Int. Cl. A61K 31/277; A61K 31/195; C07C 255/42

(52) U.S. Cl. 514/522; 514/567; 514/620; 558/410; 558/408; 562/457

(57) ABSTRACT

The invention relates to compounds of the formula (I) in which: i and j=1; R₁ is in position 3 or 4 on the phenyl ring and represents a cyano group, an alkoxy group substituted by halogen, a thioalkyl group, an alkylcarbonyl group or an alkylsulfonyl group; and R₂ represents a carboxyl group, an aikoxycarbonyl group, an alkylcarbonyl group, an unsubstituted amide group or a linear or branched alkyl group substituted by a cyano, hydroxyl, cabboxyl, aikoxycarbonyl or unsubstituted amide group; and also the pharmaceutically acceptable derivatives, salts, solvates and stereoisomers thereof, including mixtures thereof in all proportions.

The invention relates to diphenylamine derivatives, to pharmaceutical compositions comprising them and to the use thereof for the preparation of medicaments that can be used for the treatment of pathologies characterized by an oxidative stress condition, for the treatment of and preventing diabetes and/or metabolic insulin-resistance syndrome, and as a hypotriglyceridaemiant agent.

Oxidative stress is generated by many factors, for instance hyperglycaemia, dyslipidaemia (production of oxidized, highly atherogenic “low-ensity” lipoproteins (LDL)), hypoxia, insulin resistance, atherosclerosis, revascularization techniques (including angioplasties with or without a stent), chronic rejection after transplantation, the majority of inflammatory processes, and smoking. Oxidative stress is characterized at the vascular level by an increase in free radicals, in particular in superoxide anions (O₂^{.-}).

These O₂^{.-} radicals are capable of trapping the nitric oxide endogenously produced by the endothelial cells to form free-radical species that are even more deleterious, for instance peroxynitrites.

Among the pathologies concerned by an increase in oxidative stress, mention may be made of (Recent Progress in Hormone Research (1988), 53, 43-60, table V):
atherosclerosis-related ischaemias (lipid peroxidation, development, progress and rupture of atheroma plaques, platelet activation);
restenosis after angioplasty;
stenosis after vascular surgery;
diabetes;
insulin resistance;
retinal, renal and neuronal microvascular complications of diabetes, and also diabetes-related ulcers of the lower limbs;
the cardiovascular risk of diabetes that is only partially explained by the conventional factors;
male erectile dysfunction;
pulmonary arterial hypertension;
cerebral hypoxia;
chronic rejection after organ transplantation;
cold ischaemia during organ transplantation;
extracorporeal circulation;
articular pathologies.

In the context of these pathologies, an ensemble of impairments representing cardiovascular risk factors has been combined under the terrn “syndrome X” or “metabolic insulin-resistance syndrome” (MIRS) (Reaven GM: Role of insulin resistance in human disease, Diabetes 1988; 37: 1595-607); it includes insulin resistance, hyperinsulinism, glucose intolerance or declared diabetes, arterial hypertension and hypertriglyceridaemia.

Other anomalies are frequently associated with this syndrome: android obesity, microalbuminia, hyperuricaemia, clotting anomalies and fibrinolysis anomalies. Hepatic steatosis of non-alcoholic origin may also be associated therewith.

The administration of active principles capable of reducing the biological activity of oxidative free-radical species (such as superoxide anions and peroxy-nitrites) is thus particularly desirable in the treatment of these pathologies.

More specifically, the invention relates to the compounds of the formula I: 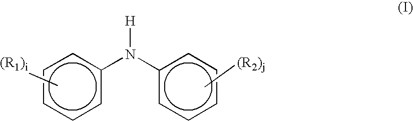
in which:
i and j=1;
R₁ is in position 3 or 4 on the phenyl ring and represents a cyano group, an alkoxy group substituted by halogen, a thioalkyl group, an alkylcarbonyl group or an alkylsulfonyl group; and
R₂ represents a carboxyl group, an alkoxycarbonyl group, an alkylcarbonyl group, an unsubstituted amide group or a linear or branched alkyl group substituted by a cyano, hydroxyl, carboxyl, alkoxycarbonyl or unsubstituted amide group, and also the pharmaceutically acceptable derivatives, salts, solvates and stereoisomers thereof, including mixtures thereof in all proportions.

A preferred subgroup of these compounds consists of compounds in which R₂ is in position 3 or 4 on the phenyl ring.

In addition, for each of the subgroups of compounds of the formula I defined above, preferred meanings of R¹, R², i and j are those listed above.

The term “halogen atom” means a fluorine, chlorine, bromine or iodine atom, preferably a fluorine or chlorine atom, in particular a fluorine atom.

The term “aliphatic hydrocarbon-based group” means a hydrocarbon-based group with a linear or branched chain containing from 1 to 14 carbon atoms, preferably from 2 to 10 carbon atoms and better still from 2 to 6 carbon atoms, for example from 2 to 4 carbon atoms.

Examples of saturated hydrocarbon-based aliphatic groups are linear or branched (C₁-C₁₀)alkyl radicals, such as methyl, ethyl, propyl, isopropyl, butyl, isobutyl, t-butyl, pentyl, isopentyl, neopentyl, 2-methylbutyl, 1-ethylpropyl, hexyl, iso-hexyl, neohexyl, 1-methylpentyl, 3-methylpentyl, 1,1-dimethylbutyl, 1,3-dimethyl-butyl, 2-ethylbutyl, 1-methyl-i-ethylpropyl, heptyl, 1-methylhexyl, 1-propylbutyl,. 4,4-dimethylpentyl, octyl, 1-methylheptyl, 2-methylhexyl, 5,5-dimethylhexyl, nonyl, decyl, 1-methylnonyl, 3,7-dimethyloctyl and 7,7-dimethyloctyl.

These alkyl groups may be substituted, especially with halogen, nitro, cyano, amino, mono- or dialkylamino, carboxyl or acylamino; alkylsulfonyl.

If the hydrocarbon-based aliphatic group is unsaturated, it may comprise one or two unsaturations. The unsaturations are of either ethylenic or acetylenic type. They are preferably ethylenic. The unsaturated chains contain at least two carbon atoms.

Alkenyl and alkynyl groups are examples of unsaturated aliphatic hydrocarbon-based groups.

Examples of unsaturated aliphatic hydrocarbon-based groups are allyl or vinyl.

The expression “optionally interrupted by O and/or S” means that any carbon atom of the hydrocarbon-based chain may be replaced with an oxygen or sulfur atom, this carbon atom not being able to be located at the free end of the hydrocarbon-based chain. The hydrocarbon-based chain, which may be alkyl, may comprise several oxygen and/or sulfur atoms, the hetero atoms preferably being separated from each other by at least one carbon atom and better still by at least two carbon atoms.

An example of an aliphatic hydrocarbon-based chain interrupted by O or S is alkoxy or thioalkoxy.

Examples of halogenated saturated hydrocarbon-based aliphatic groups are haloalkyl groups, such as perhaloalkyl groups of the type —CF₃, —CF₂—CF₃, —CCl₃ or —CCl₂CCl₃.

Similarly, an example of a halogenated alkoxy group is a perhalo group, such as trifluoromethoxy.

More generally, the substituent R₁ is chosen from halogen atoms and the following groups: cyano; carboxyl; nitro; optionally halogenated (C₁-C₁₄)alkoxy (and preferably methoxy and trifluoromethoxy); optionally halogenated (Ci—C₁₄)-thioalkoxy, preferably (C₁-C₁₀)thioalkoxy (and especially thiomethoxy); optionally halogenated and preferably perhalogenated (C₂-C₁₄)alkyl (and especially methyl and trifluoromethyl); (C₁-C₁₄)alkylcarbonyl and especially methylcarbonyl; (C₁-C₁₄)-alkoxycarbonyl and especially methoxycarbonyl and ethoxycarbonyl; di(C₁-C₁₀)alkylamino, in particular dimethylamino; and (C₁-C₁₀)alkylsulfonyl, such as methyl-sulfonyl; and (C₁-C₁₄)alkylcarbonylamino.

The substituent R₂ is advantageously cyano, a hydroxy(C₁-C₁₀)alkyl group, such as CH₂OH; a (C₁-C₁₀)alkylcarbonyl group and especially methylcarbonyl; a carboxyl or (C₁-C₆)alkylcarboxyl group, such as —CH₂COOH, an alkoxycarbonyl group, in particular —COOCH₃ or —COOC₂H₅; and an acylamino or (C₁-C₆)alkylacyl-amino group.

The two phenyl groups may be substituted one or more times with one or more of the substituents listed above, which may be identical or different, preferably one to three times, for example one or two times.

Advantageously, the compounds contain only one substituent R₁ and/or only one substituent R₂, respectively, on each of the two phenyl rings. A preferred subgroup of compounds thus consists of compounds for which i=1 and/or j=1.

The substituents R₁ and R₂ may be located on any one of the ortho, meta or para positions of the phenyl ring.

In addition, the invention relates to the optically active forms (stereoisomers), enantiomers, racemates, diastereoisomers, hydrates and solvates of these compounds. The term “solvate” denotes the adducts of the compounds with inert solvent molecules, which are formed on account of their mutual force of attraction. The solvates are, for example, the monohydrates, dihydrates or alcoholates. The term “pharmaceutically acceptable derivatives” is supposed to denote, for example, the salts of the compounds according to the invention and the compounds known as “prodrugs”.

The term “prodrugs” is defined as denoting, for example, the compounds according to formula (I) that have been modified, for example with alkyl or acyl groups, sugars or oligopeptides, and that are rapidly cleaved in the body to release the active compounds according to the invention.

They also include biodegradable polymer derivatives of the compounds according to the invention, as described, for example, in Int. J. Pharm. 115, 61-67 (1995).

The invention also relates to mixtures of the compounds of the formula (I) according to the invention, for example mixtures of two diastereoisomers, for. example in a ratio 1:1, 1:2, 1:3, 1:4, 1:5, 1:10, 1:100 or 1:1000. They are also mixtures of particularly preferred stereoisomeric compounds.

The invention is directed not only towards the compounds, but also towards the salts thereof.

If the compound of the formula II comprises an acidic function, for example a carboxylic function, this compound can form a salt with a mineral or organic base.

Examples of salts with organic or mineral bases that may be mentioned include the salts formed with metals and especially alkali metals, alkaline-earth metals and transition metals (such as sodium, potassium, calcium, magnesium or aluminium), or with bases, for instance ammonia or secondary or tertiary amines (such as diethylamine, triethylamine, piperidine, piperazine or morpholine), or with basic amino acids, or with osamines (such as meglumine) or with amino alcohols (such as 3-aminobutanol and 2-aminoethanol).

If the compound of the formula II contains a basic function, for example a nitrogen atom, this compound can form a salt with an organic or mineral acid.

The salts with organic or mineral acids are, for example, the hydrochloride, hydrobromide, sulfate, hydrogen sulfate, dihydrogen phosphate, nitrate, trifluoroacetate, citrate, maleate, fumarate, 2-naphthalenesulfonate and para-toluenesulfonate.

The invention also covers the salts allowing a suitable separation or crystallization of the compounds of the formula II, such as picric acid, oxalic acid or an optically active acid, for example tartaric acid, dibenzoyltartaric acid, mandelic acid or camphorsulfonic acid. However, a preferred subgroup of salts consists of salts of the compounds of the formula I with pharmaceutically acceptable acids or bases.

According to another aspect, and more generally, the invention relates to a pharmaceutical composition comprising a compound of the formula (II) 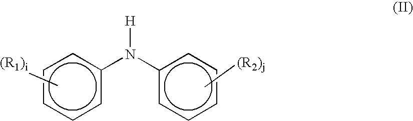
in which:
R₁ represents, independently of each other, a halogen atom; an aliphatic hydrocarbon-based group optionally substituted and/or optionally interrupted by one or more oxygen or sulfur atoms; a nitro group; a cyano group; an amino group; a mono- or dialkylamino group; an acylamino group, an alkylcarbonyl group; a carboxyl group; an unsubstituted amide group; an alkylsulfonyl group;
R₂ represents, independently of each other, a cyano group; a hydroxyl group, an alkylcarbonyl group; a carboxyl group; an alkoxycarbonyl group; an unsubstituted amide group; or a linear or branched alkyl group substituted by a cyano, hydroxyl, carboxyl, alkoxycarbonyl or unsubstituted amide group,.
i and j independently being from 1 to 5, and also the pharmaceutically acceptable derivatives, salts, solvates and stereoisomers thereof, including mixtures thereof in all proportions, in combination with a pharmaceutically acceptable excipient.

According to yet another aspect, the invention is directed towards the use of a compound of the formula II for the preparation of a medicament that can be used in the treatment of pathologies characterized by an oxidative stress condition.

More particularly, these compounds can be used for the preparation of a medicament that is useful for the treatment of and preventing diabetes and/or metabolic insulin-resistance syndrome. Moreover, they can be used for the preparation of a hypotriglyceridaemiant medicament.

It will be understood that the compounds of the formula I constitute a subgroup of the compounds of the formula II.

Thus, in the description hereinbelow, the indications given for the compounds of the formula II are also valid for the compounds of the formula I.

The compounds of the formula II can be prepared by performing one of the following processes.

Preparation of the Compounds of the Formulae I and II—Route A—

One method for the preparation of compounds of the formulae I and II consists in reacting a compound of the formula (III) with a compound of the formula (IV) 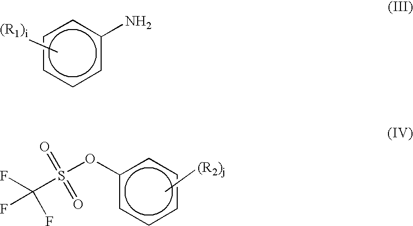
in which R₁, R₂, i and j have the meanings given above. Advantageously, it is desirable to introduce a palladium-based catalyst into the reaction medium.

Such a catalyst can be obtained by introducing into the reaction medium the system Pd(OAc)₂ +BINAP in which BINAP is the diphosphine of the formula:

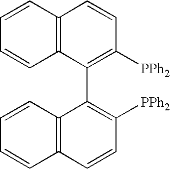

Such a catalyst can also be obtained by introducing into the reaction medium the system (dba)₃Pd₂ (tris(dibenzylideneacetone)dipalladium(0))+BINAP.

Another catalytic system may be composed of Pd(dba)₂ and tri-tert-butyl-phosphine.

By way of illustration, each of the catalytic substances is introduced into the reaction medium in a proportion of less than 10% by weight. In a particularly advantageous manner, the molar ratio of the BINAP to the (dba)₃Pd₂ or Pd(OAc)₂ ranges between 1 and 3 and preferably between 1.2 and 2.

The molar ratio between the Pd(dba)₂ and tri-tert-butylphosphine is advantageously between 1 and 3 and preferably between 1.2 and 2.

This reaction is preferably performed in the presence of an organic or mineral base. Examples of bases are hydroxides (such as alkali metal hydroxides or ammonium hydroxides), carbonates (such as alkali metal carbonates or ammonium carbonates), alkali metal alkoxides, organic hydrides, alkali metal amides, ammonia and amines, such as triethylamine, tributylamine, pyridine or N-methyl-morpholine, among which caesium carbonate or an alkali metal alkoxide is preferred.

This reaction is preferably performed in an apolar aprotic solvent, such as toluene or xylene.

The reaction temperature is set as a function of the reactivity of the species present and of the nature of the solvent used. Usually, the temperature ranges between −10° C. and 100° C. Usually, if the base used is an alkali metal or alkaline-earth metal carbonate, the process is performed at the reflux temperature of the solvent. In a particularly advantageous manner, the reaction is performed at a temperature of between 20 and 100° C.

Usually, the molar ratio of compound III to compound IV ranges between 0.8 and 2 and preferably between 0.9 and 1.5, for example between 1.0 and 1.3, a slight excess of compound III possibly being desirable.

The amount of base to be introduced into the reaction medium is generally an excess relative to the molar amount of the compound of the formula II. Preferably, the molar ratio of the base used to compound III ranges between 1 and 2 equivalents, for example between 1.3 and 1.5 equivalents. One variant comprises the reaction of a compound of the formula (III) with a compound of the formula (IX) 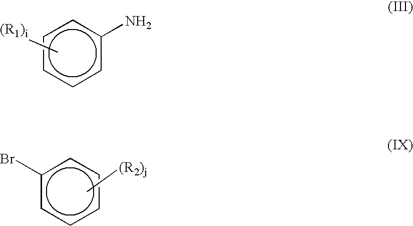
in which R₁, R₂, i and j have the meanings given above.

The reaction conditions are similar to those described above.

Preparation of the Compounds of the Formulae I and II—Route B—

Another process for the preparation of compounds of the formulae (I) and (II) comprises the reaction of a compound of the formula (V) with a compound of the formula (VI): 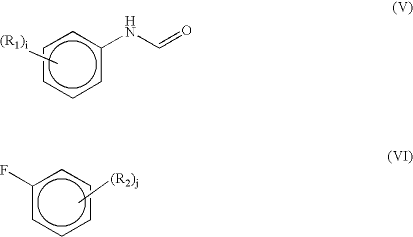
in which R₁, R₂, i and j have the meanings given above.

During this reaction, the fluoro compound VI reacts with compound V, the formyl group of which provides disubstitution. The formyl group is then removed by hydrolysis in basic medium. The base may be an alkali metal hydroxide or hydride or alternatively a base, such as lithium diisopropylamide (LDA), and in particular sodium hydride.

The reaction is advantageously performed by using an amount of base close to the stoichiometric amount. It is thus preferred to have a molar ratio of from 1 to 1.1.

This reaction is preferably performed in a polar aprotic solvent, such as a halogenated hydrocarbon (for example methylene chloride, chloroform, carbon tetrachloride, dichloroethane, chlorobenzene or dichlorobenzene); an ether, such as diethyl ether, diisopropyl ether, tetrahydrofuran, dioxane, dimethoxyethane or diethylene glycol dimethyl ether; a nitrile, such as an acetonitrile or isobutyronitrile; an amide, such as formamide, dimethylformamide, dimethylacetamide, N-methyl-2-pyrrolidinone or hexamethylphosphorylamide; or a ketone, such as acetone or 2-butanone. The solvent is preferably an amide, such as dimethylformamide.

The reaction temperature is set as a function of the reactivity of the species present and of the nature of the solvent used. The temperature usually ranges between −10° C. and 150° C. The process is usually performed at the reflux temperature of the solvent. In a particularly advantageous manner, the reaction is performed in an aprotic solvent, such as dimethylformamide at a temperature of between 120 and 140° C.

Usually, the molar ratio of compound VI to compound V ranges between 0.8 and 2 and preferably between 1 and 1.5, for example between 1.1 and 1.3, a slight excess of compound VI being desirable.

The amide thus obtained is then hydrolysed in a manner that is known per se, to give the compounds of the formulae I and II. The hydrolysis is advantageously performed in the presence of a base, such as NaOH. The hydrolysis usually proceeds satisfactorily at room temperature.

Preparation of the Compounds of the Formulae I and II—Route C—

Another process for the preparation of a compound of the formulae (I) and (II) comprises the reaction of a compound of the formula (VII) with a compound of the formula (VIII) 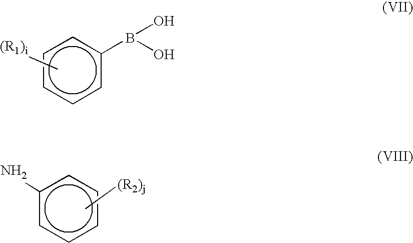
in which R₁, R₂, i and j have the meanings given above.

This reaction is preferably performed in the presence of an organic base. Examples of bases are especially alkali metal alkoxides, organic hydrides and amines, such as triethylamine, tributylamine, pyridine or N-methylmorpholine, triethylamine being particularly preferred.

The reaction takes place in the presence of copper acetate.

This reaction is preferably performed in a polar aprotic solvent, such as a halogenated hydrocarbon (for example methylene chloride, chloroform, carbon tetrachloride, dichloroethane, chlorobenzene or dichlorobenzene); an ether, such as diethyl ether, diisopropyl ether, tetrahydrofuran, dioxane, dimethoxyethane or diethylene glycol dimethyl ether; a nitrile, such as an acetonitrile or isobutyronitrile; an amide, such as formamide, dimethylformamide, dimethylacetamide, N-methyl-2-pyrrolidinone or hexamethylphosphorylamide; or a ketone, such as acetone or 2-butanone. The solvent is preferably methylene chloride.

The reaction temperature is set as a function of the reactivity of the species present and the nature of the solvent used. Usually, the reaction temperature ranges between −10° C. and 100° C. In a particularly advantageous manner, the reaction is performed at room temperature.

Usually, the molar ratio of compound VII to compound VIII ranges between 1 and 6 and preferably between 1.5 and 5, for example between 2 and 4.

The amount of base to be introduced into the reaction medium is generally equivalent to the molar amount of the compound of the formula VII.

Preparation of the Compounds of the Formulae I and II—Route D—

Yet another process for the preparation of a compound of the formulae (I) and (II) comprises the reaction of a compound of the formula (III) with a compound of the formula (X) 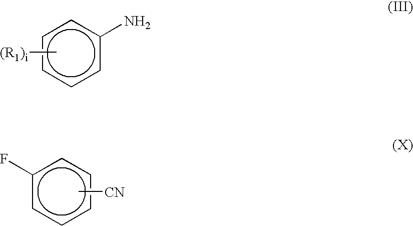
in which R₁ and j have the meanings given above.

The molar ratio of compound III to compound X is generally between 0.8 and 1.2 and preferably about 1.

The coupling is performed in the presence of an organic base chosen from those mentioned in the preceding processes. The amount of base introduced is generally an excess relative to compound III, i.e. between 1 and 2 eq. The solvent used is preferably DMSO.

The reaction temperature depends on the reactivity of the reagents and on the catalytic system used. However, it is generally possible to perform the reaction at room temperature.

The subsequent hydrolysis, under standard conditions, of the nitrile group present on the phenyl ring of the compound obtained then leads to the compounds of the formula II for which R₂ is carboxyl.

The compounds of the formula II have antioxidant activity that makes them capable of limiting the destructive activity of oxidative free-radical species.

According to yet another of its aspects, the invention thus relates to a pharmaceutical composition comprising a compound of the formula II or pharmaceutically acceptable salts thereof, in combination with a pharmaceutically acceptable excipient.

The preferred meanings of R1, R2, i and j are as described above for formula II.

These compositions can be administered orally in the form of tablets, gel capsules or granules with immediate release or controlled release, intravenously in the form of an injectable solution, transdermally in the form of an adhesive transdermal device, or locally in the form of a solution, cream or gel.

A solid composition for oral administration is prepared by adding to the active principle a filler and, where appropriate, a binder, a disintegrant, a lubricant, a colorant or a flavour corrector, and by shaping the mixture into a tablet, a coated tablet, a granule, a powder or a capsule.

Examples of fillers include lactose, corn starch, sucrose, glucose, sorbitol, crystalline cellulose and silicon dioxide, and examples of binders include poly(vinyl alcohol), poly(vinyl ether), ethylcellulose, methylcellulose, acacia, gum tragacanth, gelatin, shellac, hydroxypropylcellulose, hydroxypropylmethylcellulose, calcium citrate, dextrin and pectin. Examples of lubricants include magnesium stearate, talc, polyethylene glycol, silica and hardened plant oils. The colorant can be any colorant permitted for use in medicaments. Examples of flavour correctors include cocoa powder, mint in herb form, aromatic powder, mint in oil form, bomeol and cinnamon powder. Needless to say, the tablet or granulate may be suitably coated with sugar, gelatin or the like.

An injectable form comprising the compound of the present invention as active principle is prepared, where appropriate, by mixing the said compound with a pH regulator, a buffer agent, a suspending agent, solubilizing agent, a stabilizer, a tonicity agent and/or a preserving agent, and by converting the mixture into a form for intravenous, subcutaneous or intramuscular injection, according to a conventional process. Where appropriate, the injectable form obtained can be freeze-dried by a conventional process.

Examples of suspending agents include methylcellulose, polysorbate 80, hydroxyethylcellulose, acacia, powdered gum tragacanth, sodium carboxymethyl-cellulose and polyethoxylated sorbitan monolaurate.

Examples of solubilizing agents include castor oil solidified with polyoxyethylene, polysorbate 80, nicotinamide, polyethoxylated sorbitan monolaurate and the ethyl ester of castor oil fatty acid.

In addition, the stabilizer encompasses sodium sulfite, sodium metasulfite and ether, while the preserving agent encompasses methyl p-hydroxybenzoate, ethyl p-hydroxybenzoate, sorbic acid, phenol, cresol and chlorocresol.

The compounds of the invention of the formula II reduce the biological activity of oxidative free-radical species.

This activity is evaluated using the protocol described below.

Human LDLs placed in aqueous solution in the presence of cupric ions, become spontaneously oxidized on their protein component, apolipoprotein-B. This oxidation makes the particle fluorescent, which is exploited to measure a pharmacological effect.

The reactions and measurements are performed in black 96-well plates. 10 μl of a solution of the test product dissolved in dimethyl sulfoxide are first mixed with 170 μl of a solution of human LDL at a concentration of 120 μg/ml and 20 μl of 100 μM CuCl₂. After stirring, the mixture is incubated for 2 hours at 37° C., and a first fluorescence reading is taken (excitation at 360 nm, reading at 460 nm). The mixture is then incubated for a further 22 hours, to take a second reading under the same conditions. The difference is proportionately smaller the greater the anti-oxidant power of the test product. Probucol is used as reference product at a concentration of 10 μM.

The concentrations that inhibit 50% (IC₅₀) of the oxidation are prepared from three concentrations of the test product. They are given in Table I below for some of the compounds given as examples below.

TABLE I

| | IC₅₀ antioxidant effect |
|---|---|
| Examples | (μM) |
| 2 | 10.6 |
| 6 | 8.5 |
| 8 | 5.8 |
| 13 | 9.7 |
| 17 | 11.1 |
| 42 | 18.6 |

The compounds also show hypotriglyceridaemiant activity. This activity was especially observed by the inventors on a model of animal pathology.

The compound of Example 2 was tested on fatty Zucker rats (Zucker L. M. et al., 1961, Fatty a new mutation in the rat, J. Hered., 52: 275278). This animal is hyperphagic, obese and hyperinsulinaemic. It develops resistance to insulin, it is hyperlipidaemic, and has a large hypertriglyceridaemia. 9-week-old male Zucker rats were treated for eight days with this compound at a dose of 300 mg/kg/day p.o. After fasting for four hours, a blood sample is taken to recover the plasma. Under these conditions, the compound of Example 2 induces a large reduction in triglycerides, of 55% (p<0.01), and the insulinaemia is down by 59% (p<0.05).

The compounds of the formula II of the invention moreover have the effect of reducing the blood contents of free fatty acids and of increasing the blood contents of HDL cholesterol.

The treatment has an effect on the insulinaemia, which is lowered and allows modification of the resistance to insulin.

These properties of the compounds of the invention are useful in the prevention and treatment of diabetes, especially on account of the improvement in: the sensitivity to insulin.

The present invention is illustrated below in the light of the examples that follow.

The frequency of the NMR machine used to record the proton spectra of the examples proposed below is 300 MHz. The sign s denotes a singlet; d a doublet; t a triplet; q a quartet and m a multiplet. m.p. denotes the melting point.

The LC-MS spectra are obtained on a simple quadrupole machine equipped with an electrospray probe.

### EXAMPLES

### Example 1

### 4-[(4-Methoxyphenyl)amino]benzoic acid

a) methyl 4-[(4-methoxyphenyl)amino]benzoate

0.303 g (1.35 mmol) of palladium diacetate, 1.04 g (1.69 mmol) of racemic BINAP and then 10.25 g (31.47 mmol) of caesium carbonate are added, at room temperature, to a mixture of 6.39 g (22.5 mmol) of methyl 4-([(trifluoromethyl)sulfonyl]oxy}benzoate, prepared according to Mowery M.E. and DeShong P. (J. Org. Chem (1999) 64, 3266-3270), 3.32 g (27 mmol) of 4-methoxyaniline and 45 ml of toluene under nitrogen. The reaction medium is heated at 80° C. for 6 hours. After cooling, the reaction medium is poured into 4 l of water and extracted with ethyl ether. The organic phase is washed with water, dried over Na₂SO₄ and then concentrated to give a brown oil, which, after purification by chromatography on silica gel in CH₂Cl₂, gives 5.38 g of a beige-coloured solid.

Yield: 93.1% m.p. =88-90° C. IR (KBr): ν (NH) 3384 cm⁻¹; (CO) 1690 cm⁻¹ NMR: (CDCl₃): 3.85 (3H, s); 3.90 (3H, s); 6.9-7.1 (4H, m); 7.25 (2H, m); 7.85 (2H, m); 8.6 (1 H, s)

b) 4-[(4-methoxyphenyl)amino]benzoic acid

A mixture of 73.8 g (286 mmol) of the compound prepared in Example 1a, 590 ml of ethanol, 32.1 g (572 mmol) of KOH and 290 ml of water is refluxed for 2 hours.

The reaction medium is then concentrated, taken up in 1600 ml of water, washed with 3×250 ml of ethyl ether and filtered, and is then acidified with acetic acid. The precipitate formed is washed with water (3×250 ml) and dried under vacuum to give 66.6 g of a pink-white solid. After recrystallization from an ethyl acetate/heptane mixture, 55.3 g of a pink-white solid are obtained.

Yield: 79.5% m.p.=162-164° C. IR (KBr): ν (NH) 3402 cm⁻¹; (CO) 1673 cm⁻¹ NMR: (DMSO-d6): 3.75 (3H, s); 6.8-7.0 (4H, m); 7.1 (2H, m); 7.7 (2H, m); 8.4 (1H, s, exchangeable with CF₃COOD); 12.2 (1H, broad s, exchangeable with CF₃COOD). LC-MS:
(ES+)=244.2 (M+H)
(ES−)=242.1 (M−H)

### Example 2

### 4-[(4-Methoxyphenyl)amino]benzoic acid

A solution of 3 g (19.8 mmol) of 4-methoxyphenylformamide, prepared from 4-methoxyaniline according to Ugi I. and Meyr R. (Org. Syntheses, Coll. Vol. 5, 1060-1063), in 7 ml of dimethylformamide (DMF), is added dropwise slowly, between 10 and 20° C., to a suspension of 0.87 g (21.8 mmol) of NaH at 60% in liquid petroleum jelly, in 3 ml of DMF. After stirring for 30 minutes at room temperature, a solution of 3.5 g (20.7 mmol) of ethyl 4-fluorobenzoate in 5 ml of DMF is added dropwise. The reaction medium is heated at 130° C. for 22 hours. After cooling, 3 ml of 1ON HCl solution are added and the reaction medium is concentrated to dryness under vacuum. 40 ml of ethanol, 10 ml of water, 10 ml of THF and 10.6 ml of aqueous 30% NaOH solution are added to the residue obtained. After stirring for 16 hours at room temperature, the reaction medium is concentrated under vacuum. The residue is taken up in 30 ml of water, washed with CH₂Cl₂ (3×30 ml) and acidified to pH 7 with 1ON HCl solution. The precipitate formed is filtered off, washed with water and dried under vacuum to give 1.64 g of a beige-coloured solid, which is identical to the product obtained in Example lb.

Yield: 34%

### Example 3

### 4-[(4-Methoxyphenyl)amino]benzoic acid

a) ethyl 4-[(4-methoxyphenylamino]benzoate

b 0.597 g (3.3 mmol) of copper (II) acetate is added to a solution of 0.545 g (3.3 mmol) of ethyl 4-aminobenzoate in 20 ml of CH₂Cl₂, followed by addition of 1 g (6.6 mmol) of 4-methoxyphenylboronic acid and 0.92 ml (6.6 mmol) of triethylamine. After stirring for 18 hours at room temperature, a further 1.19 g (6.6 mmol) of copper (II) acetate, 1 g (6.6 mmol) of 4-methoxyphenylboronic acid and 0.92 ml (6.6 mmol) of triethylamine are added, and stirring is then continued at room temperature for 24 hours. The reaction medium is then poured into water and extracted with CH₂Cl₂. The organic phase is washed with water, dried over Na₂SO₄ and then concentrated and purified by flash chromatography on silica with, a (6/1) heptane/ethyl acetate mixture, to give 0.543 g of a beige-coloured solid.

Yield: 60.7% IR (KBr): ν (NH): 3344 cm⁻¹; (CO): 1697 cm⁻¹ NMR: (DMSO-d6): 1.15 (3H, t, J=7.1 Hz); 3.6 (3H, s); 4.1 (2H, q, J=7.1 Hz); 6.8 (4H, m); 7.0 (2H, m); 7.6 (2H, m); 8.4 (1 H, s, exchangeable with D₂0)

b) 4-[(4-mnethoxyphenyl) amino]benzoic acid

Obtained by working as in Example 1 b, starting with the compound obtained in Example 3a.

### Example 4

### 4-[(4-Methoxyphenyl)amino]benzoic acid

a) ethyl 4-[(4-methoxyphenyl)amino]benzoate

1.76 ml (0.176 mmol) of a 0.1 M solution of tri-tert-butylphosphine in toluene are added to a mixture composed of 2.52 g (11 mmol) of ethyl 4-bromobenzoate, 1.354 g (11 mmol) of 4-methoxyaniline and 0.128 g (0.22 mmol) of bis(dibenzylideneacetone)palladium (0) in 20 ml of toluene, followed by addition of 1.58 g (16.5 mmol) of sodium tert-butoxide. After stirring for 20 hours at room temperature, the reaction medium is taken up in water and extracted with ethyl ether. The organic phase is washed with water, dried over Na₂SO₄ and concentrated under vacuum. The residue obtained is purified by flash chromatography on silica in a (1/1) heptane/ethyl acetate mixture, to give 1.96 g of a beige-coloured solid, the NMR characteristics of which are identical to those of the product of Example 3a.

Yield: 65.8%

b) 4-[(methoxyphenyl)amino]benzoic acid

Obtained by working as in Example 1b.

### Example 5

### 4-{[4-(Trifluoromethyl)phenyl)amino]benzoic acid

a) ethyl 4-{[4-(trifluoromethyl)phenyl]amino}benzoate

A mixture composed of 9.1 g (28 mmol) of caesium carbonate, 0.458 g (0.5 mmol) of tris(dibenzylideneacetone)dipalladium, 0.934 g (1.5 mmol) of racemic BINAP (2,2-bis(diphenylphosphino)-1,1-binaphthyl), 4.5 g (20 mmol) of 1-bromo-4-(trifluoromethyl)benzene, 3.96 g (28 mmol) of ethyl 4-aminobenzoate and 60 ml of diglyme (diethylene glycol dimethyl ether) is heated at 100° C. for 6 hours.

After cooling, the reaction medium is poured into water and extracted with ether. The organic phase is washed with water, dried over Na₂SO₄ and concentrated under vacuum. The residue obtained is purified by flash chromatography on silica in an (11/9) dichloromethane/hexane mixture, to give 5.5 g of a pale yellow solid.

Yield: 89% IR (KBr): ν (NH): 3334 cm⁻¹; (CO): 1682 cm^{-1,} 1699 cm⁻¹ NMR: (CDCl₃): 1.35 (3H, t, J=7.1 Hz); 4.3 (2H, q, J=7.1 Hz); 6.15 (1H, broad s, exchangeable with D₂O); 7.0 (2H, m); 7.1 (2H, m); 7.5 (2H, m); 7.9 (2H, m)

b) 4-{[4-trifluoromethyl)phenyl]amino}benzoic acid

Obtained by working as in Example 1b.

Yield: 76.6% IR (KBr): ν (NH): 3415 cm⁻¹; (CO): 1670 cm⁻¹ NMR: (DMSO-d6): 7.4 (2H, m); 7.5 (2H, m); 7.8 (2H, m); 8.0 (2H, m); 9.35 (1H, s, exchangeable with CF₃COOD); 12.7 (1H, broad s, exchangeable with CF₃COOD).

### Example 6

### 4-[(4-Methoxyphenyl)amino]benzonitrile

1.7 g (15 mmol) of sodium tert-butoxide are added to a mixture composed of 1.21 g (10 mmol) of 4-fluorobenzonitrile, 1.23 g (10 mmol) of 4-methoxyaniline and 10 ml of DMSO. The reaction medium is stirred for 24 hours at room temperature, and then poured into 120 ml of water and extracted with ether. The organic phase is washed with water until neutral, dried over Na₂SO₄ and concentrated under vacuum. The residue obtained is purified by flash chromatography on silica, in a (1/1) heptane/dichloromethane mixture, to give 0.882 g of a pale yellow solid.

Yield: 39% NMR: (DMSO-d6): 3.55 (3H, s); 6.65-6.8 (4H, m); 6.9 (2H, m); 7.3 (2H, m); 8.5 (1H, s)

This compound was also obtained by working as in Example 2a, in a yield of 61.9%.

### Example 7

### 4-[(4-Methoxyphenyl)amino]benzoic acid

A mixture of 3 g (13.4 mmol) of 4-[(4-methoxyphenyl)amino]benzonitrile, obtained in Example 6a, 1.5 g (26.8 mmol) of KOH and 80 ml of ethylene glycol is refluxed for 4 hours. After cooling, the mixture is poured into ice-cold water and acidified with acetic acid. The precipitate formed is filtered off by suction, washed with water and dried under vacuum. 2.9 g of a beige-coloured solid having the same spectral (IR, NMR) properties as the compound obtained in Example lb, are obtained.

Yield: 89.2%

### Example 8

### {4-[(4-Methoxyphenyl)amino]phenyl}methanol

A solution of 1 g (3.7 mmol) of ethyl 4-[(4-methoxyphenyl)amino]benzoate, obtained in Example 3a, in 10 ml of THF is added dropwise to a suspension of 0.21 g (5.5 mmol) LiAlH₄ in 15 ml of THF. The reaction medium is then refluxed for 2 hours. After cooling, 1 ml of ethyl acetate is added dropwise and the mixture is then hydrolysed by dropwise addition of water, and 20 ml of ether are finally added. The precipitate formed is filtered off and rinsed with ether. The filtrate is concentrated under vacuum and the residue obtained is purified by flash chromatography on silica in a (4/1) and then (1/1) heptane/ethyl acetate mixture, to give 0.34 g of a pink solid.

Yield: 40.3% m.p.=110° C. NMR: (DMSO-d6): 3.9 (3H, s); 4.57 (2H, d, J=5.6 Hz); 5.15 (1H, t, J=5.6 Hz); 7.05 (4H, m); 7.20 (2H, m); 7.25 (2H, m); 7.95 (1 H, s). NMR: (DMSO-d6): 3.80 (3H, 2s); 4.54 (2H, 2d, J=5.9 Hz, transforms 2s with CF₃COOD); 5.30 (1 H, 2t, J=5.9 Hz, exchangeable with CF₃COOD); 6.9 (4H, m); 7.15-7.6 (4H, m)

### Examples 9 TO 29

The compounds of Examples 9 to 29 were obtained as in Example 1. Their structure and characteristics are collated in Table 1.

TABLE 1

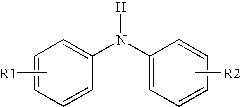

| Ex | | R1 | R2 | NMR |
|---|---|---|---|---|
| 9 | 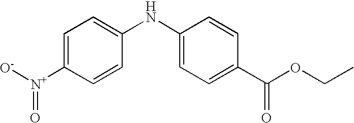 | 4-NO2 | 4-CO2Et | (CDCl3): 1.36 (3H, t, J = 7.2 Hz); 4.35 (2H, q, J = 7.2 Hz); 6.47 (1H, broad s); 6.99-7.32 (4H, m); 7.92-8.30 (4H, m). |
| 10 | 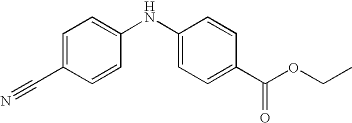 | 4-CN | 4-CO2Et | (CDCl3): 1.39 (3H, t, J = 7.2 Hz); 4.35 (2H, q, J = 7.2 Hz); 6.29 (1H, broad s); 7.02-7.20 (4H, m); 7.44-7.67 (2H, m); 7.88-8.14 (2H, m). |
| 11 | 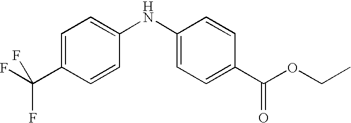 | 4-CF3 | 4-CO2Et | (CDCl3): 1.36 (3H, t, J = 7.2 Hz); 4.33 (2H, q, J = 7.2 Hz); 6.22 (1H, broad s); 7.00-7.22 (4H, m); 7.44-7.63 (2H, m); 7.82-8.11 (2H, m). |
| 12 | 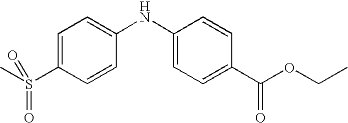 | 4-MeSO2 | 4-CO2Et | (CDCl3): 1.39 (3H, t, J = 7.2 Hz); 4.36 (2H, q, J = 7.2 Hz); 3.04 (3H, s); 6.39 (1H, s); 7.05-7.38 (4H, m); 7.67-8.20 (4H, m). |
| 13 | 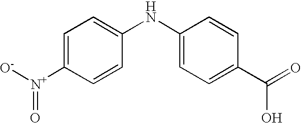 | 4-NO2 | 4-CO2H | (DMSO-d6): 7.06-7.46 (4H, m): 7.81-8.28 (4H, m); 9.62 (1H, broad s). |
| 14 | 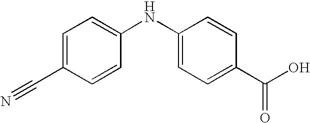 | 4-CN | 4-CO2H | (DMSO-d6): 6.92-7.44 (4H, m); 7.48-8.07 (4H, m); 9.30 (1H, broad s); 12.59 (1H, broad s). |
| 15 | 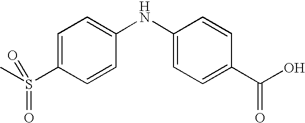 | 4-MeSO2 | 4-CO2H | (DMSO-d6): 3.13 (3H, s); 7.05-7.55 (4H, m); 7.55-8.11 (4H, m); 9.29 (1H, s); 12.51 (1H, broad s). |
| 16 | 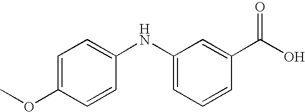 | 4-MeO | 3-CO2H | (DMSO-d6): 3.72 (3H, s); 6.82-6.95 (2H, m); 6.98-7.16 (3H, m); 7.18-7.30 (2H, m); 7.39-7.52 (1H, m); 8.04 (1H, s); 12.73 (1H, broad s). |
| 17 | 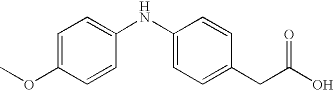 | 4-MeO | 4-CH2—CO2H | (DMSO-d6): 3.40 (2H, s); 3.70 (3H, s); 6.75-6.93 (4H, m); 6.93-7.14 (4H, m); 7.78 (1H, s); 12.16 (1H, broad s). |
| 18 | 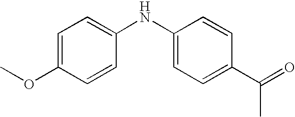 | 4-MeO | 4-COMe | (DMSO-d6): 2.41 (3H, s); 3.73 (3H, s); 6.75-7.04 (4H, m); 7.04-7.22 (2H, m); 7.63-7.99 (2H, m); 8.55 (1H, s). |
| 19 | 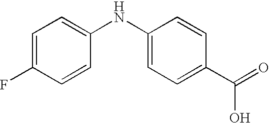 | 4-F | 4-COOH | (DMSO-d6): 6.66-7.46 (6H, m); 7.55-8.03 (2H, m); 8.65 (1H, s); 12.29 (1H, broad s). |
| 20 | 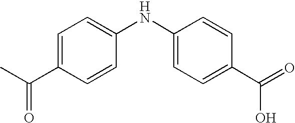 | 4-MeCO | 4-COOH | (DMSO-d6): 2.49 (3H, s); 6.91-7.46 (4H, m); 7.64-8.01 (4H, m); 9.23 (1H, s); 12.50 (1H, broad s). |
| 21 | 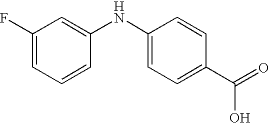 | 3-F | 4-COOH | (DMSO-d6): 6.59-7.46 (6H, m); 7.68-7.97 (2H, m); 8.90 (1H, s); 12.39 (1H, broad s). |
| 22 | 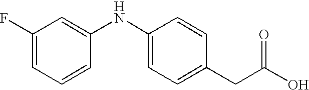 | 3-F | 4-CH2—CO2H | (DMSO-d6): 3.47 (2H, s); 6.33-6.93 (3H, m); 6.93-7.34 (5H,m); 8.35 (1H, s); 12.2 (1H, broad s). |
| 23 | 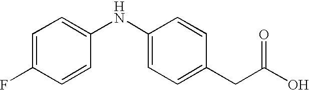 | 4-F | 4-CH2—CO2H | (DMSO-d6): 3.43 (2H, s); 6.78-7.22 (8H, m); 8.04 (1H, s); 12.20 (1H, broad s). |
| 24 | 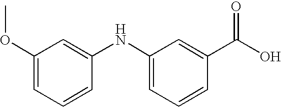 | 3-MeO | 3-CO2H | (DMSO-d6): 3.71 (3H, s); 6.30-6.85 (3H, m); 6.96-7.54 (4H, m); 7.54-7.73 (1H, m); 8.37 (1H, s); 12.83 (1H, broad s). |
| 25 | 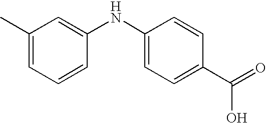 | 3-Me | 4-CO2H | (DMSO-d6): 2.27 (3H, s); 6.50-7.48 (6H, m); 7.57-7.98 (2H, m); 8.63 (1H, s); 12.83 (1H, broad s). |
| 26 | 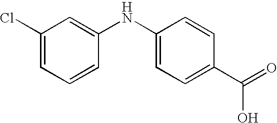 | 3-Cl | 4-CO2H | (DMSO-d6): 6.77-7.52 (6H, m); 7.52-8.21 (2H, m); 8.87 (1H, s); 12.40 (1H, broad s). |
| 27 | 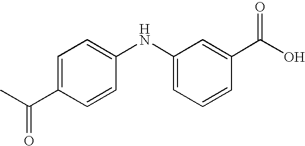 | 4-MeCO | 3-CO2H | (DMSO-d6): 2.49 (3H, s); 6.80-8.17 (8H, m); 9.01 (1H, s); 12.99 (1H, broad s). |
| 28 | 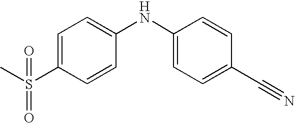 | 4-MeSO2 | 4-CN | (DMSO-d6): 3.14 (3H, s); 7.09-7.46 (4H, m); 7.54-7.91 (4H, m); 9.43 (1H, s). |
| 29 | 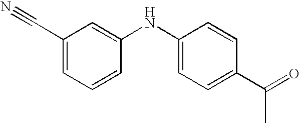 | 3-CN | 4-COMe | (DMSO-d6): 2.49 (3H, s); 7.05-7.63 (6H, m); 7.79-7.99 (2H, m); 9.11 (1H, s). |

### Example 30

### 4,4′-Iminodibenzoic acid

Obtained by working as in Example 7a, starting with 4-[(4-cyanophenyl)-amino]benzoic acid (Example 14).

Yield: 89.4% IR (KBr): ν (NH): 3404 cm⁻¹; (CO): 1667 cm⁻¹ NMR: (DMSO-d6): 7.2 (4H, m); 7.85 (4H, m); 9.2 (1H, s, exchangeable with D₂O); 12.5 (1 H, broad s, exchangeable with CF₃COOD)

### EXAMPLES 31 to 52

The compounds of Examples 31 to 52 were obtained as in Example 1a or 5a, and then lb or 8. Their structure and characteristics are collated in Table 2. The NMR spectra in Table 2 were acquired in DMSOd6.

TABLE 2

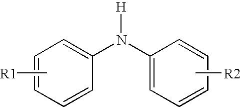

| Ex | | R1 | R2 | MR |
|---|---|---|---|---|
| 31 | 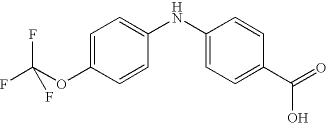 | 4-F3CO | 4-CO2H | 7.07 (d, J=8.77 Hz, 2 H) 7.25 (m, 4 H) 7.79 (d, J=8.77 Hz, 2 H) 8.86 (s, 1 H) 12.35 (s, 1 H) |
| 32 | 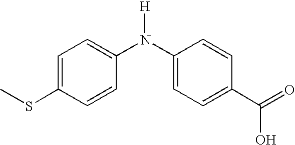 | 4-MeS | 4-CO2H | 2.44 (s, 3 H) 7.14 (m, 6 H) 7.76 (d, J=8.58 Hz, 2 H) 8.70 (s,1 H) 12.29 (s, 1 H) |
| 33 | 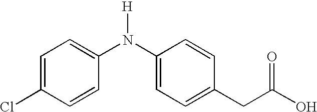 | 4-Cl | 4-CH2—CO2H | 3.46 (s, 2 H) 7.09 (m, 8 H) 8.24 (s, 1 H) 12.20 (s, 1 H) |
| 34 | 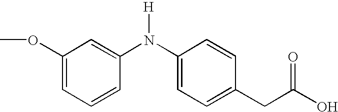 | 3-MeO | 4-CH2—CO2H | 3.45 (s, 2 H) 3.69 (s, 3 H) 6.37 (dd, J=8.20, 1.72 Hz, 1 H) 6.59 (m, 2 H) 7.06 (m, 5 H) 8.11 (s, 1 H) 12.21 (s, 1 H) |
| 35 | 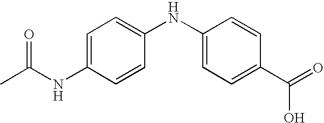 | 4-AcNH | 4-CO2H | 2.01 (s, 3 H) 6.94 (d, J=8.77 Hz, 2H) 7.10 (d, J=8.77 Hz, 2H) 7.51 (d, J=8.77 Hz, 2H) 7.74 (d, J=8.77 Hz, 2H) 8.57 (s, 1 H) 9.85 (s, 1 H) 12.22 (s, 1 H) |
| 36 | 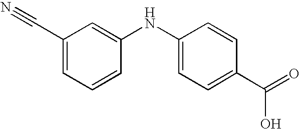 | 3-CN | 4-CO2H | 7.34 (m, 6 H) 7.83 (d, J=8.58 Hz, 2 H) 9.01 (s, 1 H) 12.46 (s, 1 H) |
| 37 | 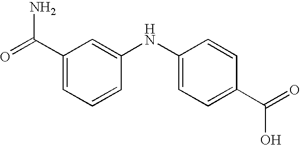 | 3-CN | 4-CO2H | 3.54 (s, 3 H) 7.25 (m, 5 H) 7.75 (m, 3 H) 8.65 (s, 1 H) |
| 38 | 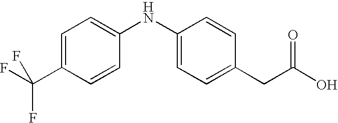 | 4-CF3 | 4-CH2—CO2H | 3.49 (s, 2 H) 7.14 (m, 6 H) 7.48 (d, J=8.58 Hz, 2 H) 8.63 (s, 1 H) 12.28 (s, 1 H) |
| 39 | 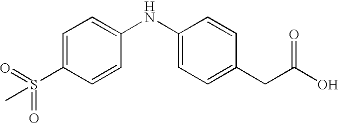 | 4-MeSO2 | 4-CH2—CO2H | 3.08 (s, 3 H) 3.51 (s, 2 H) 7.14 (m, 6 H) 7.67 (d, J=8.77 Hz, 2 H) 8.83 (s, 1 H) 12.29 (s, 1 H) |
| 40 | 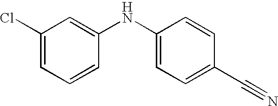 | 3-Cl | 4-CN | 7.14 (m, 6 H) 7.63 (m, 2 H) 9.05 (s, 1 H) |
| 41 | 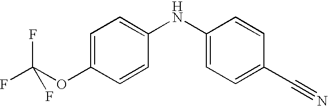 | 4-CF3O | 4-CN | 7.24 (m, 6 H) 7.61 (d, J=8.58 Hz, 2 H) 9.03 (s, 1 H) |
| 42 | 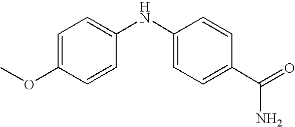 | 4-MeO | 4-CONH2 | 3.72 (s, 3 H) 6.87 (m, 5 H) 7.09 (d, J=8.67 Hz, 2 H) 7.61 (s, 1 H) 7.68 (d, J=8.29 Hz, 2 H) 8.24 (s, 1 H) |
| 43 | 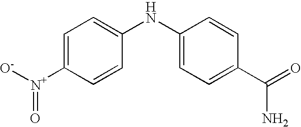 | 4-NO2 | 4-CONH2 | 7.20 (m, 5 H) 7.86 (m, 3 H) 8.12 (m, 2 H) 9.51 (s, 1 H) |
| 44 | 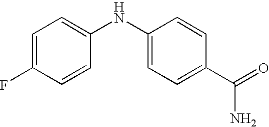 | 4-F | 4-CONH2 | 7.08 (m, 7 H) 7.73 (m, 3 H) 8.47 (s, 1 H) |
| 45 | 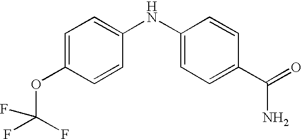 | 4-CF3O | 4-CONH2 | 7.17 (m, 7 H) 7.75 (m, 3 H) 8.70 (s, 1 H) |
| 46 | 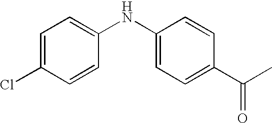 | 4-Cl | 4-COMe | 2.45 (s, 3 H) 7.22 (m, 6 H) 7.83 (d, J=8.77 Hz, 2 H) 8.90 (s, 1 H) |
| 47 | 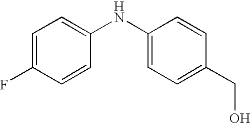 | 4-F | 4-CH2OH | 4.37 (d, J=5.53 Hz, 2 H) 4.97 (m, 1 H) 7.05 (m, 8 H) 8.02 (s, 1 H) |
| 48 | 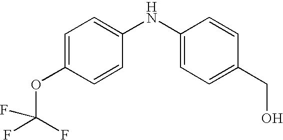 | 4-CF3O | 4-CH2OH | 4.40 (d, J=5.53 Hz, 2 H) 5.01 (m, 1 H) 7.11 (m, 8 H) 8.28 (s, 1 H) |
| 49 | 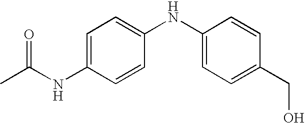 | 4-NHAc | 4-CH2OH | 3.32 (s, 3 H) 4.36 (d, J=5.53 Hz, 2 H) 4.94 (d, J=5.53 Hz, 1 H) 7.00 (m, 6 H) 7.40 (m, 2 H) 7.93 (s, 1 H) 9.72 (s, 1 H) |
| 50 | 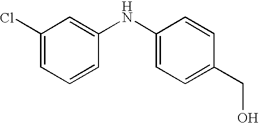 | 3-Cl | 4-CH2OH | 4.41 (d, J=5.53 Hz, 2 H) 5.03 (t, J=5.53 Hz, 1 H) 6.77 (m, 1 H) 7.08 (m, 7 H) 8.32 (s, 1 H) |
| 51 | 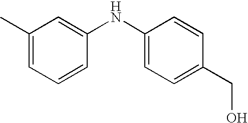 | 3-Me | 4-CH2OH | 2.22 (s, 3 H) 4.38 (d, J=5.53 Hz, 2 H) 4.97 (t, J=5.63 Hz, 1 H) 6.60 (d, J=7.25 Hz, 1 H) 7.02 (m, 7 H) 7.99 (s, 1 H) |
| 52 | 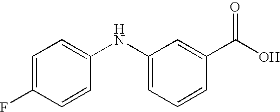 | 4-F | 3-CO2H | 7.31 (m, 8 H) 8.38 (s, 1 H) 12.90 (s, 1 H) |
| 53 | 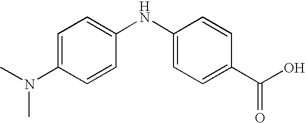 | 4-(NMe2) | 4-COOH | 2.8 (s, 6 H) 6.8 (m, 4 H) 7.0(m, 2 H) 7.7 (d, J=8.8 Hz, 2 H) 8.3 (s, 1H) 12.1(s, 1H) |
| 54 | 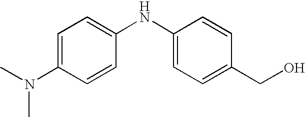 | 4-(NMe2) | 4-CH2OH | 2.8 (s, 6 H) 4.3 (d J=5.5 Hz, 2 H) 4.9 (t, J=5.5 Hz, 1 H) 6.7 (m.2 H) 6.8 (m, 2 H) 7.0 (m, 2H) 7.1 (m, 2 H) 7.6 (s, 1H) |

1. Compounds of the formula (I) 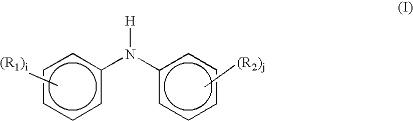
in which:
i and j=1;
R₁ is in position 3 or 4 on the phenyl ring and represents a cyano group, an alkoxy group substituted by halogen, a thioalkyl group, an alkylcarbonyl group or an alkylsulfonyl group; and
R₂ represents a carboxyl group, an alkoxycarbonyl group, an alkylcarbonyl group, an unsubstituted amide group or a linear or branched alkyl group substituted by a cyano, hydroxyl, carboxyl, alkoxycarbonyl or unsubstituted amide group; and also the pharmaceutically acceptable derivatives, salts, solvates and stereoisomers thereof, including mixtures thereof in all proportions.

2. Compounds of the formula (I) according to claim 1, in which R₂ is in position 3 or 4 on the phenyl ring.

3. Compounds of the formula (I) according to claim 1, in which R₁ is chosen from the group consisting of —CN, —F, —Cl, —SO₂CH₃, —COCH₃, —OCF₃ and —SCH₃ groups.

4. Compounds of the formula (I) according to claim 1, in which R₂ is chosen from the group consisting of —CO₂C₂H₅, —COOH, —CH₂COOH, —CN, —COCH₃, —CONH₂ and —CH₂OH groups.

5. Compounds of the formula (I) according to claim 1, chosen from the following compounds:
4-[(4-methoxyphenyl)amino]benzoic acid;
{4-[(4-methoxyphenyl)amino]phenyl}methanol;
ethyl 4-[(4-cyanophenyl)amino]benzoate;
ethyl 4-[[4-(methylsulfonyl)phenyl]amino]benzoate;
4-[(4-cyanophenyl)amino]benzoic acid;
4-[[4-(methylsulfonyl)phenyl]amino]benzoic acid;
3-[(4-methoxyphenyl)amino]benzoic acid;
{4-[(4-methoxyphenyl)amino]phenyl}acetic acid;
1-{4-[(4-methoxyphenyl)amino]phenyl }ethanone;
4-[(4-fluorophenyl)amino]benzoic acid;
4-[(4-acetylphenyl)amino]benzoic acid;
4-[(3-fluorophenyl)amino]benzoic acid;
{4-[(3-fluorophenyl)amino]phenyl}acetic acid;
{4-[(4-fluorophenyl)amino]phenyl}acetic acid;
3-[(3-methoxyphenyl)amino]benzoic acid;
4-[(3-chlorophenyl)amino]benzoic acid;
3-[(4-acetylphenyl)amino]benzoic acid;
4-[[4-(methylsulfonyl)phenyl]amino]benzonitrile;
3-[(4-acetylphenyl)amino]benzonitrile;
4-{[4-(trifluoromethoxy)phenyl]amino}benzoic acid;
4-[[4-(methylthio)phenyl]amino]benzoic acid;
{4-[(4-chlorophenyl)amino]phenyl}acetic acid;
{4-[(3-methoxyphenyl)amino]phenyl}acetic acid;
4-[(3-cyanophenyl)amino]benzoic acid;
4-[[3-(aminocarbonyl)phenyl]amino]benzoic acid;
{4-[[4-(methylsulfonyl)phenyl]amino]phenyl} acetic acid;
4-[(3-chlorophenyl)amino]benzonitrile;
4-{[4-(trifluoromethoxy)phenyl]amino}benzonitrile;
4-[(4-methoxyphenyl)amino]benzamide;
4-[(4-nitrophenyl)amino]benzamide; 4-[(4-fluorophenyl)amino]benzamide;
4-{[4-(trifluoromethoxy)phenyl]amino}benzamide;
1-{4-[(4-chlorophenyl)amino]phenyl}ethanone;
{4-[(4-fluorophenyl)amino]phenyl}methanol;
(4-{[4-(trifluoromethoxy)phenyl]amino}phenyl)methanol;
{4-[(3-chlorophenyl)amino]phenyl}methanol;
3-[(4-fluorophenyl)amino]benzoic acid;
4-{[4-(dimethylamino)phenyl]amino}benzoic acid;
(4-{[4-{dimethylamino)phenyl]amino}phenyl)methanol.

6. Pharmaceutical composition comprising a compound of the formula (II) 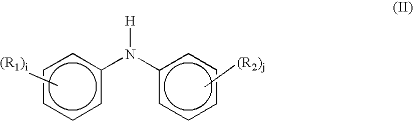
in which:
R₁ represents, independently of each other, a halogen atom; an aliphatic hydrocarbon-based group optionally substituted and/or optionally interrupted by one or more oxygen or sulfur atoms; a nitro group; a cyano group; an amino group; a mono- or dialkylamino group; an al kylcarbonyl group; a carboxyl group; an alkylcarbonylamino group; an alkylsulfonyl group;
R₂ represents, independently of each other, a cyano group; an hydroxyl group, an alkylcarbonyl group; a carboxyl group; an alkoxycarbonyl group; an unsubstituted amide group; or a linear or branched alkyl group substituted by a cyano, hydroxyl, carboxyl, alkoxycarbonyl or unsubstituted amide group.
i and j independently being from 1 to 5,
and also the pharmaceutically acceptable derivatives, salts, solvates and stereoisomers thereof, including mixtures thereof in all proportions in combination with a pharmaceutically acceptable excipient.

7. Composition according to claim 6, in which R₂ is in position 3 or 4 on the phenyl ring.

8. Composition according to claim 6, in which R₁ is chosen from the group consisting of —CN, —F, —Cl, —SO₂CH₃, —OCH₃, —COCH₃, —OCF₃, —SCH₃, —CF₃, —NO₂, —CH₃, —COOH, —CN, —CONH₂, —NHCOCH₃ and —N(CH₃)₂ groups.

9. Composition according to claim 6, in which R₂ is chosen from the group consisting of —CO₂C₂H₅, —COOH, —CH₂COOH, —CN, —COCH₃, —CONH₂ and —CH₂OH groups.

10. Composition according to claim 6, in which the compound of the formula (II) is chosen from the following compounds:
4-[(4-methoxyphenyl)amino]benzoic acid;
4-{[4-(trifluoromethyl)phenyl]amino}benzoic acid;
4-[(4-methoxyphenyl)amino]benzonitrile;
{4-[(4-methoxyphenyl)amino]phenyl}methanol;
ethyl 4-[(4-nitrophenyl)amino]benzoate;
ethyl 4-[(4-cyanophenyl)amino]benzoate;
ethyl 4-{[4-(trifluoromethyl)phenyl]amino}benzoate;
ethyl 4-[[4-(methylsulfonyl)phenyl]amino]benzoate;
4-[(4-nitrophenyl)amino]benzoic acid;
4-[(4-cyanophenyl)amino]benzoic acid;
4-[[4-(methylsulfonyl)phenyl]amino]benzoic acid;
3-[(4-methoxyphenyl)amino]benzoic acid;
{4-[(4-methoxyphenyl)amino]phenyl}acetic acid;
1-{4-[(4-methoxyphenyl)amino]phenyl}ethanone;
4-[(4-fluorophenyl)amino]benzoic acid;
4-[(4-acetylphenyl)amino]benzoic acid;
4-[(3-fluorophenyl)amino]benzoic acid;
{4-[(3-fluorophenyl)amino]phenyl}acetic acid;
{4-[(4-fluorophenyl)amino]phenyl}acetic acid;
3-[(3-methoxyphenyl)amino]benzoic acid;
4-[(3-methylphenyl)amino]benzoic acid;
4-[(3-chlorophenyl)amino]benzoic acid;
3-[(4-acetylphenyl)amino]benzoic acid;
4-[[4-(methylsulfonyl)phenyl]amino]benzonitrile;
3-[(4-acetylphenyl)amino]benzonitrile;
4,4′-iminodibenzoic acid;
4-{[4-(trifluoromethoxy)phenyl]amino}benzoic acid;
4-[[4-(methylthio)phenyl]amino]benzoic acid;
{4-[(4-chlorophenyl)amino]phenyl}acetic acid;
{4-[(3-methoxyphenyl)amino]phenyl}acetic acid;
4-[[4-(acetylamino)phenyl]amino]benzoic acid;
4-[(3-cyanophenyl)amino]benzoic acid;
4-[[3-(aminocarbonyl)phenyl]amino]benzoic acid;
(4-{[4-(trifluoromethyl)phenyl]amino}phenyl)acetic acid;
{4-[[4-(methylsulfonyl)phenyl]amino]phenyl}acetic acid;
4-[(3-chlorophenyl)amino]benzonitrile;
4-{[4-(trifluoromethoxy)phenyl]amino}benzonitrile;
4-[(4-methoxyphenyl)amino]benzamide;
4-[(4-nitrophenyl)amino]benzamide;
4-[(4-fluorophenyl)amino]benzamide;
4-{[4-(trifluoromethoxy)phenyl]amino}benzamide;
{4-[(4-chlorophenyl)amino]phenyl}ethanone;
{4-[(4-fluorophenyi)amino]phenyl}methanol;
(4-{[4-(trifluoromethoxy)phenyl]amino}phenyl)methanol;
N-{4-[[4-(hydroxymethyl)phenyl]amino]phenyl}acetamide;
{4-[(3-chlorophenyl)amino]phenyl}methanol;
{4-[(3-methylphenyl)amino]phenyl}methanol;
3-[(4-fluorophenyl)amino]benzoic acid;
4-{[4-(dimethylamino)phenyl]amino}benzoic acid;
(4-{[4-{dimethylamino)phenyl]amino}phenyl)methanol.

11. Use of a compound of the formula (II) according to claim 6, for the preparation of a medicament that is useful for the treatment of pathologies characterized by an oxidative stress condition.

12. Use of a compound of the formula (II) according to claim 6, for the preparation of a medicament that is useful for the treatment of and preventing diabetes and/or metabolic insulin-resistance syndrome.

13. Use of a compound of the formula (II) according to claim 6, for the preparation of a hypotriglyceridaemiant medicament.

14. Use of a compound of the formula (II) according to claim 6, for the preparation of an antioxidant medicament that can be used as a free-radical scavenger.
